(19) United States
(12) Patent Application Publication
(10) Pub. No.: US 2022/0304713 A1
(43) Pub. Date: Sep. 29, 2022

(54) SURGICAL INSTRUMENTS INCLUDING ADAPTER ASSEMBLY

(71) Applicant: Covidien LP, Mansfield, MA (US)

(72) Inventors: Justin J. Thomas, New Haven, CT (US); Roy J. Pilletere, Middletown, CT (US); Saumya Banerjee, Hamden, CT (US); Jacob C. Baril, Norwalk, CT (US); Garrett P. Ebersole, Hamden, CT (US); Matthew A. DiNino, Newington, CT (US)

(21) Appl. No.: 17/590,838

(22) Filed: Feb. 2, 2022

Related U.S. Application Data
(60) Provisional application No. 63/164,622, filed on Mar. 23, 2021.

(51) Int. Cl. A61B 17/28

(52) U.S. Cl. CPC A61B 17/2841; A61B 2017/00473

(57) ABSTRACT

A surgical kit includes a handle assembly, an adapter assembly, and at least one end effector. The adapter assembly allows for use of a handle assembly that is configured to affect a function of a relatively long end effector to also be used with a relatively short end effector.

### CROSS-REFERENCE TO RELATED APPLICATION

The present application claims the benefit of and priority to U.S. Provisional Patent Application Ser. No. 63/164,622, filed on Mar. 23, 2021, the entire content of which being hereby incorporated by reference.

### BACKGROUND

### Technical Field

The disclosure relates to surgical instruments including an adapter assembly. More specifically, the disclosure relates to surgical instruments having an adapter assembly for allowing a handle assembly to be used with end effectors of different lengths.

### Background of Related Art

Surgical instruments often include a handle assembly that is configured to control a function of an end effector assembly that is directly or indirectly engaged therewith. End effectors are typically available in different lengths depending on the desired surgical function. When a particular handle assembly does not include a reliable way to limit the longitudinal travel of a drive rod, for instance, it may be suitable to provide end effectors having one length to be used with such a handle assembly to avoid damaging portions of the surgical instrument, for instance.

### SUMMARY

The disclosure relates to a surgical kit including a handle assembly, an adapter assembly, and a first end effector. The handle assembly includes a handle portion, an elongated portion extending distally from the handle portion, an actuation switch, and a firing rod. At least a portion of the firing rod extends through the elongated portion. Actuation of the actuation switch causes the firing rod to move distally relative to the handle portion. The adapter assembly is configured to selectively engage the handle assembly and includes a connector, an adapter drive rod, and a biasing element. A proximal portion of the connector is configured to engage a distal portion of the firing rod of the handle assembly. A proximal portion of the adapter drive rod is coupled to a distal portion of the connector. The biasing element is disposed between a distal end of the connector and a portion of the adapter drive rod. The first end effector defines a first length, is configured to selectively engage the adapter assembly, and includes a drive assembly and a jaw member. A proximal portion of the drive assembly is configured to engage a distal portion of the adapter drive rod.

In disclosed embodiments, the adapter assembly and the handle assembly are configured such that when the adapter assembly is engaged with the handle assembly and with the first end effector, actuation of the actuation switch causes a first distal movement of the firing rod, a corresponding first distal movement of the adapter drive rod, and a corresponding first distal movement of the drive assembly of the first end effector to a distal-most position relative to the jaw member. It is also disclosed that the handle assembly is configured such that the actuation of the actuation switch causes a corresponding first distal movement of the connector of the adapter assembly. In embodiments, the handle assembly is configured such that the actuation of the actuation switch causes a second distal movement of the firing rod, a corresponding second distal movement of the connector of the adapter assembly, and results in the drive assembly of the first end effector remaining in its longitudinal position. It is further disclosed the adapter assembly is configured such that that the adapter drive rod remains in its longitudinal position in response to the second distal movement of the firing rod, and that the adapter assembly and the handle assembly are configured such that the second distal movement of the firing rod causes the biasing element to compress the amount of the second distal movement of the firing rod.

Additionally, it is disclosed that the biasing element of the adapter assembly is disposed between the distal end of the connector and a proximal-facing shoulder of the adapter drive rod.

In embodiments, the surgical kit also includes a second end effector defining a second length and configured to selectively engage the adapter assembly. The second length is different from the first length.

It is also disclosed that the adapter assembly is configured such that the connector of the adapter assembly is longitudinally translatable relative to the drive assembly of the first end effector.

In disclosed embodiments, the adapter assembly and the handle assembly are configured such that when the adapter assembly is engaged with the handle assembly, the connector is longitudinally fixed relative to the firing rod.

It is disclosed that the biasing element of the adapter assembly is a compression spring.

The disclosure also relates to a surgical instrument comprising a handle assembly, an adapter assembly, and an end effector. The handle assembly includes an actuation switch and a firing rod. The adapter assembly is configured to selectively engage the firing rod, and includes a connector, an adapter drive rod, and a biasing element. The connector is configured to engage a distal portion of the firing rod of the handle assembly. The biasing element is configured to bias the adapter drive rod distally relative to the connector. The end effector is configured to selectively engage the adapter assembly, and includes a drive assembly and a jaw member. The drive assembly is configured to engage a distal portion of the adapter drive rod. Actuation of the actuation switch causes the firing rod and the connector to move distally.

In disclosed embodiments, initial actuation of the actuation switch causes the drive assembly of the end effector to move distally.

It is also disclosed that the connector of the adapter assembly is longitudinally translatable relative to the drive assembly of the end effector.

In embodiments, the biasing element is disposed between a distal end of the connector and a proximal-facing shoulder of the adapter drive rod.

It is further disclosed that when the adapter assembly is engaged with the handle assembly, the connector is longitudinally fixed relative to the firing rod.

The disclosure also relates to an adapter assembly for interconnecting a handle assembly of a surgical instrument and an end effector of a surgical instrument. The adapter assembly includes a connector, an adapter drive rod, and a biasing element. The connector is configured to engage a distal portion of a firing rod of the handle assembly. The adapter drive rod is disposed in mechanical cooperation with a distal portion of the connector. The biasing element is disposed between a distal-most end of the connector and a portion of the adapter drive rod.

In disclosed embodiments, the biasing element and the adapter drive rod are co-axial, and the biasing element is a compression spring.

According to another aspect of the disclosure, a surgical kit is provided and includes a handle assembly having a handle portion; an elongated portion extending distally from the handle portion; an actuation switch; and a firing rod, at least a portion of the firing rod extending through the elongated portion. In use, actuation of the actuation switch causes the firing rod to move distally relative to the handle portion. The surgical kit further includes an adapter assembly configured to selectively engage the handle assembly. The adapter assembly includes a connector, a proximal portion of the connecter configured to engage a distal portion of the firing rod of the handle assembly; an adapter drive rod, a proximal portion of the adapter drive rod is coupled to a distal portion of the connector; and a biasing element disposed between a distal end of the connector and a portion of the adapter drive rod. The surgical kit still further includes a first end effector defining a first length and configured to selectively engage the adapter assembly. The first end effector includes a drive assembly, a proximal portion of the drive assembly configured to engage a distal portion of the adapter drive rod; and a jaw member.

The adapter assembly and the handle assembly may be configured such that when the adapter assembly is engaged with the handle assembly and with the first end effector, actuation of the actuation switch causes a first distal movement of the firing rod, a corresponding first distal movement of the adapter drive rod, and a corresponding first distal movement of the drive assembly of the first end effector to a distal-most position relative to the jaw member.

The handle assembly may be configured such that the actuation of the actuation switch causes a corresponding first distal movement of the connector of the adapter assembly.

The handle assembly may be configured such that the actuation of the actuation switch causes a second distal movement of the firing rod, a corresponding second distal movement of the connector of the adapter assembly, and results in the drive assembly of the first end effector remaining in its longitudinal position.

The adapter assembly may be configured such that the adapter drive rod remains in its longitudinal position in response to the second distal movement of the firing rod.

The adapter assembly and the handle assembly may be configured such that the second distal movement of the firing rod causes the biasing element to compress the amount of the second distal movement of the firing rod.

The biasing element of the adapter assembly may be disposed between the distal end of the connector and a proximal-facing shoulder of the adapter drive rod.

The surgical kit may further include a second end effector defining a second length and configured to selectively engage the adapter assembly, wherein the second length is different from the first length.

The adapter assembly may be configured such that the connector of the adapter assembly is longitudinally translatable relative to the drive assembly of the first end effector.

The adapter assembly and the handle assembly may be configured such that when the adapter assembly is engaged with the handle assembly, the connector is longitudinally fixed relative to the firing rod.

The biasing element of the adapter assembly may be a compression spring.

The biasing element and the adapter drive rod may be co-axial.

In use, initial actuation of the actuation switch may cause the drive assembly of the first end effector to move distally.

The biasing element may be configured to bias the adapter drive rod distally relative to the connector.

The first end effector may include a second jaw member pivotably disposed relative to the jaw member.

Further details and aspects of exemplary embodiments of the disclosure are described in more detail below with reference to the appended figures.

### BRIEF DESCRIPTION OF THE DRAWINGS

Embodiments of the disclosure are described herein with reference to the accompanying drawings, wherein:

FIG. 1 is a perspective view of a surgical instrument including a handle assembly, an adapter assembly, and a first end effector in accordance with an embodiment of the disclosure;

FIG. 2 is an assembly view of the surgical instrument of FIG. 1 and further including a second end effector;

FIG. 3 is an enlarged view of the area of detail indicated in FIG. 2 illustrating the adapter assembly;

FIG. 4 is a perspective view of the adapter assembly of FIG. 3;

FIG. 5 is an exploded view of the adapter assembly of FIGS. 3 and 4;

FIG. 6 is a perspective view of a drive assembly of the first end effector shown in FIG. 1;

FIG. 7 is a perspective view of the adapter assembly of FIGS. 3 and 4 with parts omitted;

FIG. 8 is a cross-sectional view of the adapter assembly taken along line 8-8 in FIG. 7;

FIG. 9 is a perspective view of the adapter assembly of FIGS. 3 and 4 with parts removed, and engaged with a distal portion of the handle assembly and with a proximal portion of the first end effector;

FIG. 10 is a cross-sectional view of the first end effector with its jaw members in an open position, the adapter assembly, and a portion of the handle assembly taken along line 10-10 of FIG. 1 prior to actuation of the handle assembly;

FIG. 11 is an enlarged view of the area of detail indicated in FIG. 10;

FIG. 12 is an enlarged view of the area of detail indicated in FIG. 10;

FIG. 13 is a cross-sectional view of the first end effector, the adapter assembly, and a portion of the handle assembly of the surgical instrument of FIG. 1 near the completion of a firing stroke of the handle assembly;

FIG. 14 is an enlarged view of the area of detail indicated in FIG. 13;

FIG. 15 is an enlarged view of the area of detail indicated in FIG. 13; and

FIG. 16 is a cross-sectional view of the adapter assembly of FIGS. 3 and 4 engaged with a distal portion of the handle assembly and with a proximal portion of the first end effector, at the completion of a firing stroke of the handle assembly.

### DETAILED DESCRIPTION

Embodiments of the disclosed surgical instrument with adapter assembly are described in detail with reference to the drawings, in which like reference numerals designate identical or corresponding elements in each of the several views. Non-limiting examples of surgical instruments and associated handle assemblies according to the disclosure include manual, robotic, mechanical and/or electromechanical surgical staplers, forceps, tack appliers (e.g., tackers), clip appliers, and the like. As used herein the term “distal” refers to that portion of the surgical instrument, or component thereof, farther from the user, while the term “proximal” refers to that portion of the surgical instrument, or component thereof, closer to the user.

As will be described in greater detail below, the disclosure includes a surgical kit including a handle assembly, an adapter assembly, and at least one end effector. The adapter assembly allows a handle assembly that is configured to affect a function of a relatively long end effector to be used with a relatively short end effector.

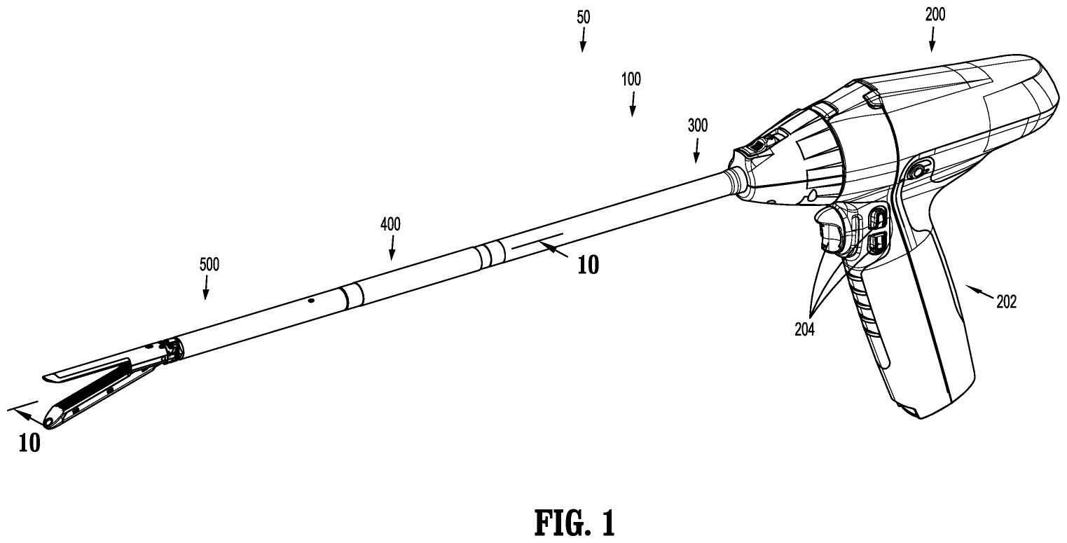

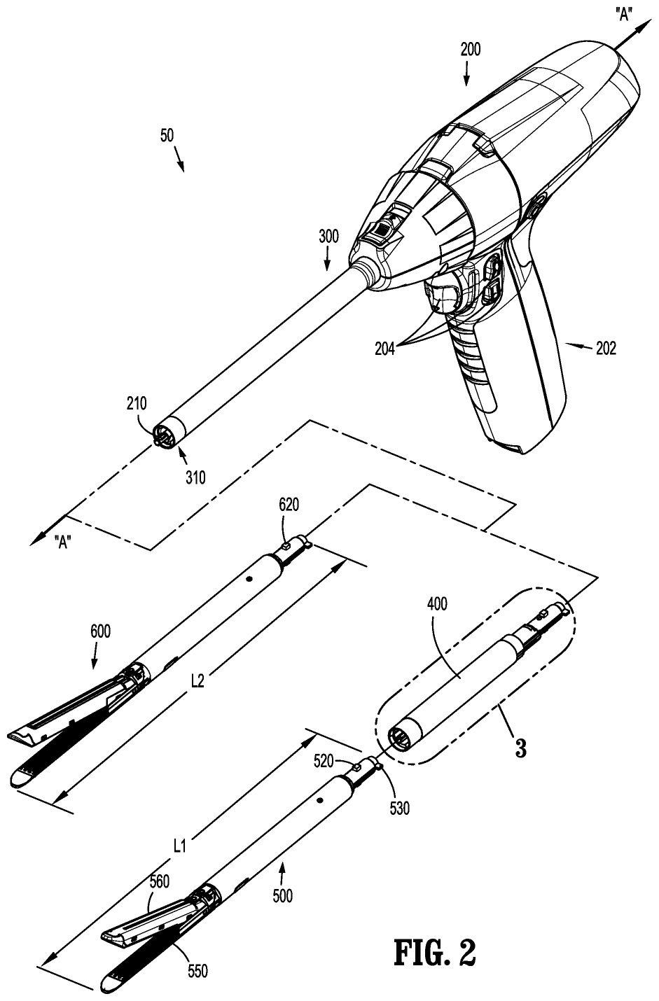

FIGS. 1 and 2 illustrate a surgical kit in accordance with an embodiment of the disclosure generally designated as reference numeral 50. The surgical kit 50 includes a handle assembly 200, an adapter assembly 400, a first end effector 500, and/or a second end effector 600 (FIG. 2). Additionally, end effectors of different lengths, for instance, may also be included in the surgical kit 50. The handle assembly 200 includes an elongated portion 300 extending distally therefrom and defining a longitudinal axis “A-A.” While the illustrated handle assembly 200 is in the form of a powered hand-held electromechanical handle, other types of handle assemblies are usable with the elongated portion 300, the adapter assembly 400, the first end effector 500, and the second end effector 600. As shown, the first end effector 500 has a shorter length “L1” than the length “L2” of the second end effector 600. Additionally, a surgical instrument 100 may include the handle assembly 200, the adapter assembly 400, the first end effector 500 and/or the second effector 600.

With continued reference to FIGS. 1 and 2, the handle assembly 200 includes a handle portion 202, and a plurality of actuation switches 204. Activation of at least one actuation switch of the plurality of actuation switches 204 is configured to advance and/or retract a firing rod 210, shown extending through the elongated portion 300 in FIG. 2. The advancement and retraction of the firing rod 210 can be accomplished with at least one motor and/or by mechanical means.

As shown in FIG. 2, the elongated portion 300 of the handle assembly 200 is configured to selectively engage the adapter assembly 400 or the second end effector 600. (The physical properties of the elongated portion 300, the adapter assembly 400, the first end effector 500, and the second end effector 600 also allow the elongated portion 300 to directly engage the first end effector 500, and allow the second end effector 600 to engage the adapter assembly 400.)

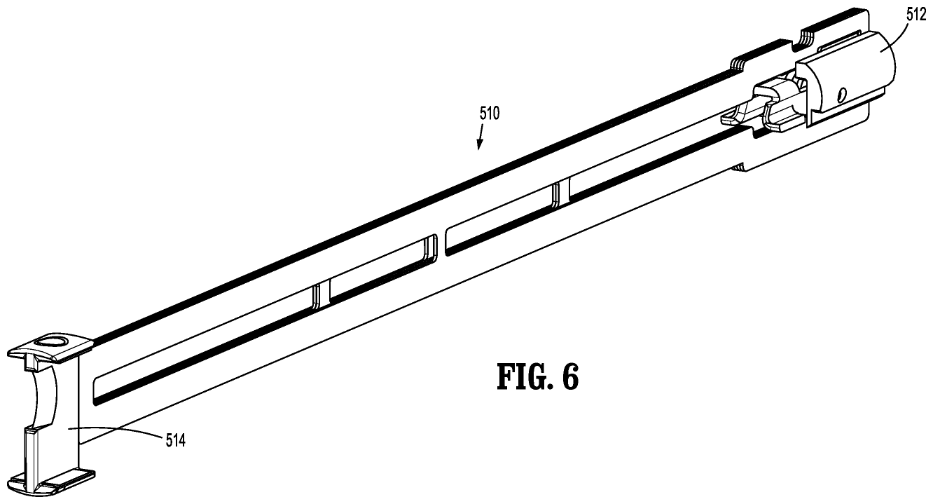

In use, the handle assembly 200 may be configured such that actuation of one actuation switch of the plurality of actuation switches 204 is configured to advance the firing rod 210 a particular distance. In embodiments, this distance is sufficient to advance a drive assembly of the longer, second end effector 600 an appropriate distance to eject all of the fasteners therein, for example. However, when the same handle assembly 200 is used with the short, first end effector 500, without the adapter assembly 400, the distance advanced by the firing rod 210 and thus a drive assembly 510 (FIG. 6) of the first end effector 500, is too great a distance and may cause damage to the first end effector 500, for instance. As described below, the adapter assembly 400 is usable with the handle assembly 200 to limit the amount of distal advancement of the drive assembly 510, while still ensuring the drive assembly 510 travels a sufficient distance to ensure a sled 516 of the end effector 500 can reliably eject the fasteners, for example.

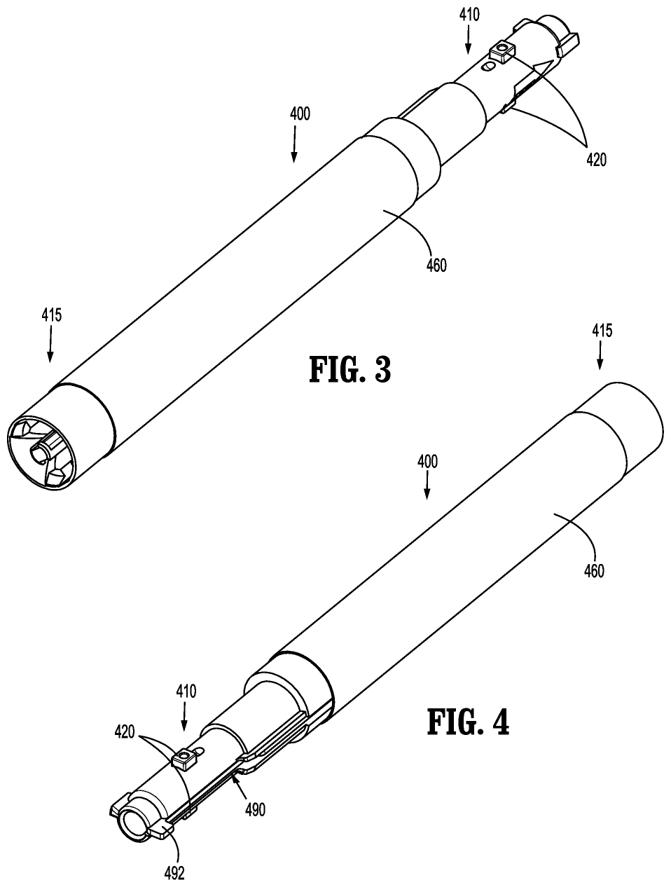

FIGS. 3-5 illustrate details of the adapter assembly 400. The adapter assembly 400 includes a proximal portion 410 configured for selective engagement with a distal portion 310 of the elongated portion 300, a distal portion 415 configured for selective engagement with a proximal portion of an end effector or loading unit (either the first end effector 500, the second end effector 600, or a different end effector), and an adapter drive assembly 430 configured to engage both the firing rod 210, which extends through the elongated portion 300, and the drive assembly (e.g., drive assembly 510 of FIG. 6) of the end effector (e.g., the first end effector 500) engaged therewith.

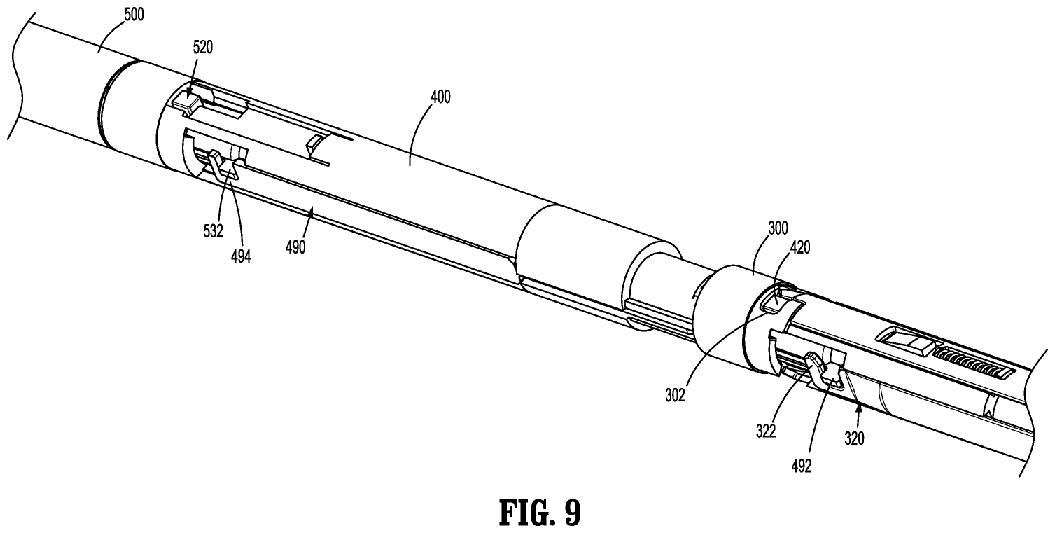

More particularly, the proximal portion 410 of the adapter assembly 400 includes at least one lug 420 configured to selectively engage a notch 302 (or other suitable structure) of the elongated portion 300 (FIG. 9). The distal portion 415 of the adapter assembly 400 includes at least one notch 422 (see FIG. 8), which is configured to selectively engage at least one lug 520, 620 (FIG. 2) of the end effectors 500, 600, engaged therewith. Moreover, the at least one lug 520, 620 of the respective end effectors 500, 600 is also configured to selectively engage the notch 302 (or other suitable structure) of the elongated portion 300, thereby allowing the adapter assembly 400 to be bypassed if desired.

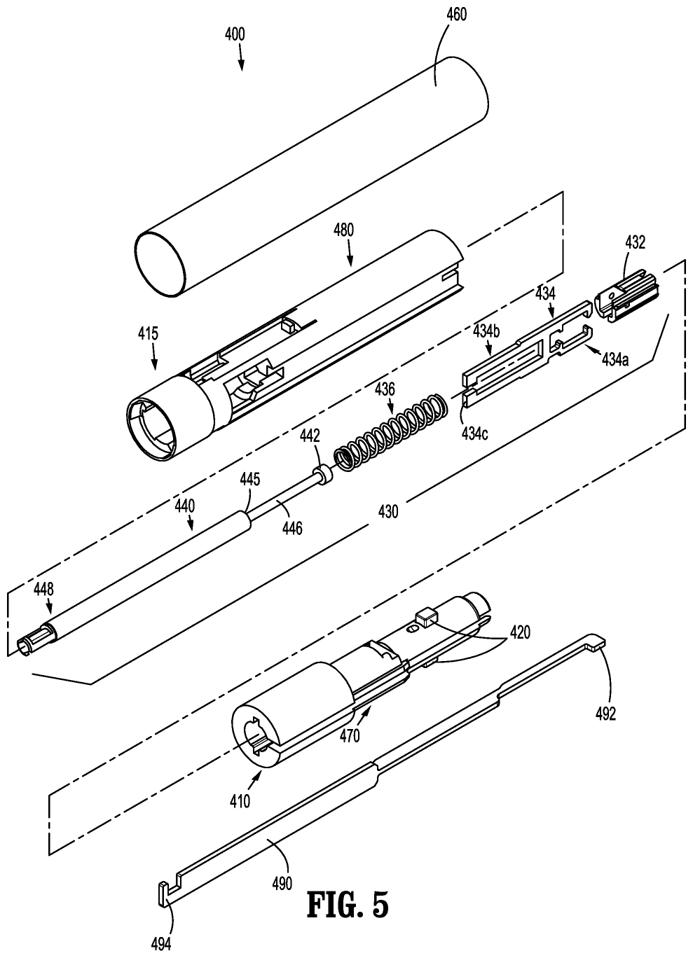

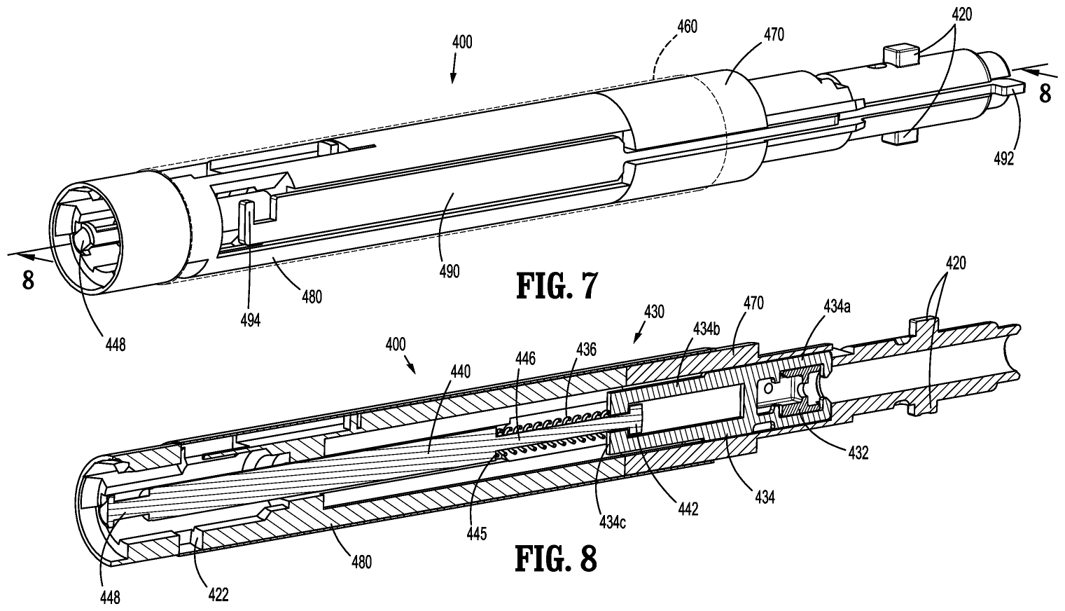

With particular reference to FIGS. 5 and 7, additional details of the adapter assembly 400 are shown. The adapter assembly 400 includes the adapter drive assembly 430, an outer tube 460, a proximal body 470, a distal body 480, and an adapter articulation link 490. The proximal body 470 and the distal body 480 are disposed at least partially within the outer tube 460. The adapter articulation link 490 extends at least partially within the outer tube 460 between the proximal portion 410 and the distal portion 415 of the adapter assembly 400. A proximal portion 492 of the articulation link 490 is configured to selectively engage a distal portion 322 of a handle articulation link 320 extending at least partially through the elongated portion 300, and a distal portion 494 of the articulation link 490 is configured to selectively engage a proximal portion 532 of an end effector articulation link 530 of the end effector 500 engaged therewith (see FIG. 9). The handle assembly 200 is usable to advance and/or retract the handle articulation link 320, which, in turn, causes the articulation link 490 of the adapter assembly 400 to correspondingly advance and/or retract, which, in turn, causes corresponding advancement/retraction of the end effector articulation link 530, which causes jaw members 550 and 560 of the end effector 500 to pivot about the longitudinal axis “A-A.”

Referring to FIGS. 5 and 8, the adapter drive assembly 430 is slidably disposed at least partially within the proximal body 470 and the distal body 480 of the adapter assembly 400. The adapter drive assembly 430 includes a proximal receptor 432, a connector 434, a biasing element 436, and an adapter drive rod 440. A proximal portion 434a of the connector 434 is configured to mechanical engage the proximal receptor 432, and a distal portion 434b of the connector 434 is configured to mechanically engage a proximal foot 442 of the adapter drive rod 440. As shown in FIG. 8, the biasing element 436 (e.g., compression spring) is positioned between a distal end 434c of the connector 434, and a proximally-facing shoulder 445 of the adapter drive rod 440. Moreover, the biasing element 436 radially surrounds a portion of a proximal shaft 446 of the adapter drive rod 440; the proximal shaft 446 extends between the proximal foot 442 and the proximally-facing shoulder 445 of the adapter drive rod 440. A distal end 448 of the adapter drive rod 440 is configured to mechanically engage an end effector receptor 512 (FIG. 6) of the drive assembly 510 of the end effector 500 (or end effector 600) engaged therewith. Moreover, the distal end 448 of the adapter drive rod 440 is configured to mechanically engage any suitable end effector (e.g., end effectors having a receptor similar to the end effector receptor 512 shown in FIG. 6).

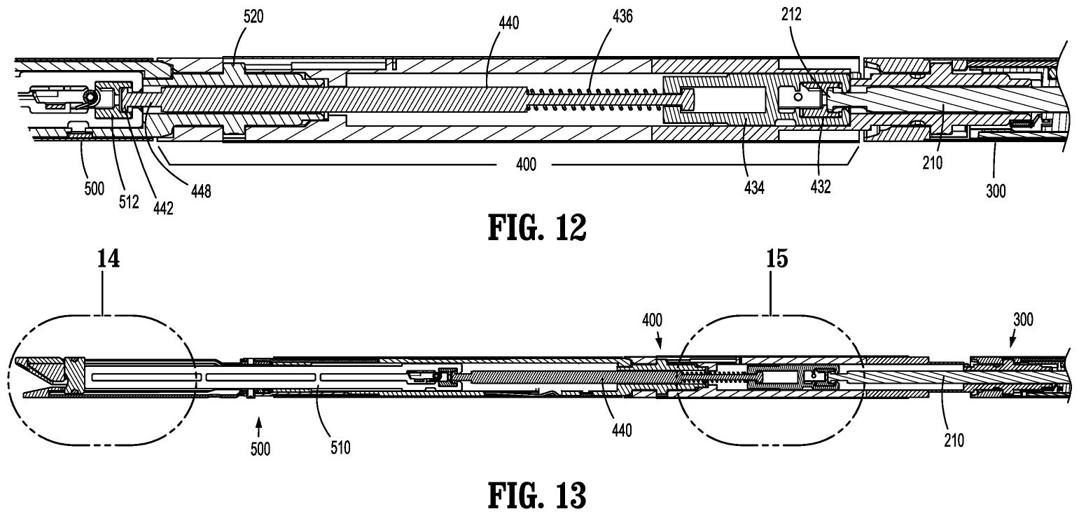

As shown in FIG. 12, when the adapter assembly 400 is engaged with the elongated portion 300, a distal finger 212 of the firing rod 210 is positioned (e.g., longitudinally fixed) within the proximal receptor 432 of the adapter assembly 400 (e.g., in response to a rotational engagement between the adapter assembly 400 and the elongated portion 300), and is thus engaged with the proximal portion 434a of the connector 434. Additionally, and as also shown in FIG. 12, when the adapter assembly 400 is engaged with the end effector 500, a distal finger 442 of the distal end 448 of the adapter drive rod 440 is positioned (e.g., longitudinally fixed) within the end effector receptor 512 of the end effector 500 (e.g., in response to a rotational engagement between the adapter assembly 400 and the end effector 500).

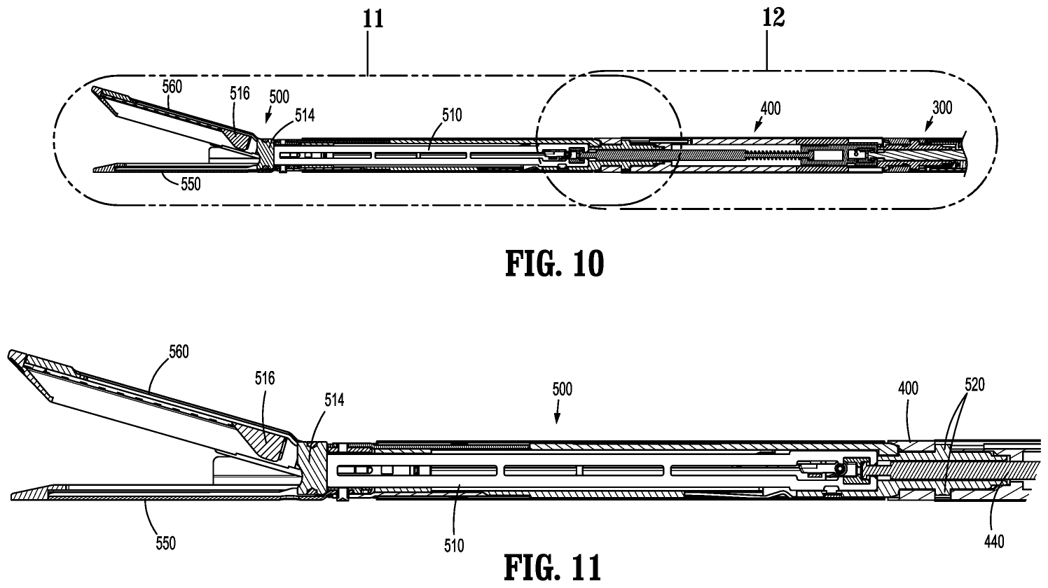

FIGS. 10-16, illustrate details of portions of the surgical kit 50, including the adapter assembly 400 and the first end effector 500, during use. With initial reference to FIGS. 10-12, the adapter assembly 400 is shown engaged with the end effector 500 and with the elongated portion 300, while the jaw members 550, 560 of the end effector 500 are in the initial, open position. Here, the actuation switch 204 of the handle assembly 200 configured to advance the firing rod 210 has not been actuated. Accordingly, the firing rod 210 is in its proximal-most position. As shown, when the firing rod 210 is in its proximal-most position, the adapter drive rod 440 is in its proximal-most position, and the drive assembly 510 of the end effector 500 are also in their proximal-most positions.

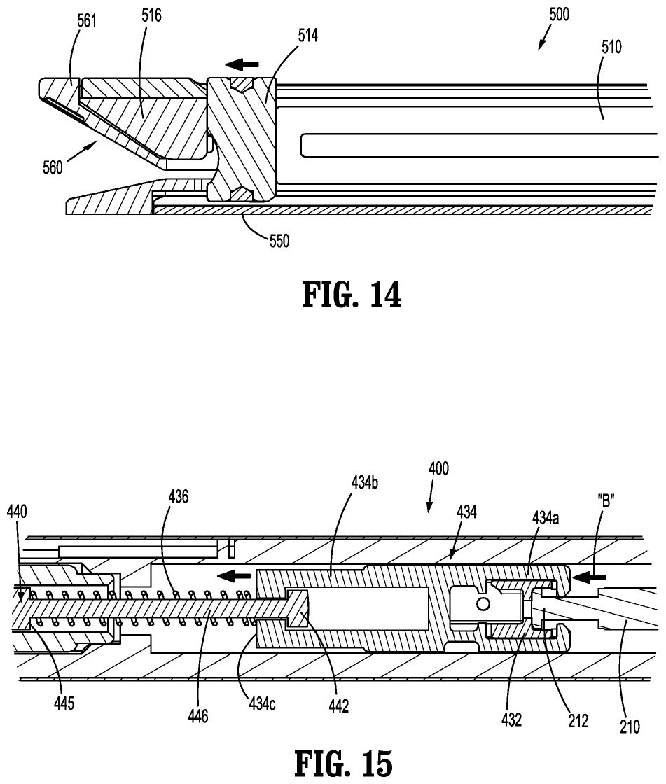

FIGS. 13-15 illustrate components of the elongated portion 300, the adapter assembly 400, and the end effector 500 corresponding to when the actuation switch 204 of the handle assembly 200 has been actuated to advance the firing rod 210. Here, the actuation of the actuation switch 204 causes distal movement of the firing rod 210 in the general direction of arrow “B” in FIG. 15. The distal movement of the firing rod 210 causes a corresponding distal movement of the connector 434 of the adapter assembly 400 due the engagement between the distal finger 212 of the firing rod 210 and the proximal receptor 432 of the adapter assembly 400. The distal movement of the connector 434 then causes the distal end 434c of the connector 434 to exert a distally-directed force against the biasing element 436. Prior to the I-beam 514 and the sled 516 of the drive assembly 510 reaching their distal-most position, the distally-directed force against the biasing element 436 is not significantly opposed, thereby causing the biasing element 436 to remain virtually uncompressed and allowing the biasing element 436 to move distally. This distal movement of the biasing element 436 results in distal movement of the drive assembly 510. Thus, in response to actuation of the actuation switch 204, the drive assembly 510 moves distally until it reaches its distal-most position, as shown in FIG. 14.

Here, while the drive assembly 510 of the end effector 500 has already reached its distal-most position, the firing rod 210 has not yet reached its distal-most position. That is, the drive assembly 510 reaches its distal-most position before the firing rod 210 reaches its distal-most position.

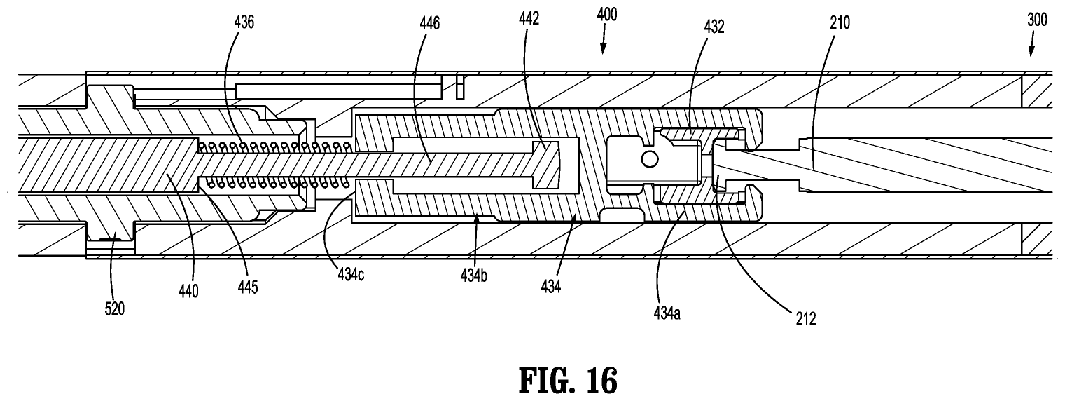

As shown in FIG. 16, the additional distal movement of the firing rod 210, as it completes its full stroke, is absorbed by the adapter assembly 400 after the drive assembly 510 has reached its distal-most position (FIG. 14). Here, the distal movement of the firing rod 210 still causes a corresponding distal movement of the connector 434 of the adapter assembly 400, and a distal end 444 of the connector 434 still exerts a distally-directed force against the biasing element 436. However, since this distally-directed force against the biasing element 436 is opposed by the engagement between the sled 516 and a distal wall 561 of the jaw member 560 (FIG. 14), the biasing element 436 is caused to compress, as shown in FIG. 16. The compression of the biasing element 436 allows the connector 434 of the adapter assembly 400 to move distally with respect to the drive assembly 510 of the end effector 500, while allowing the longitudinal position of the drive assembly 510 of the end effector 500 to remain unchanged (or substantially unchanged), thereby preventing undue force from being exerted on the drive assembly 510 and on the jaw member 560 after the drive assembly 510 has reached its distal-most position.

The longer, second end effector 600 can be directly engaged with the elongated portion 300 of the handle assembly 200, or can be directly engaged with the adapter assembly 400. In embodiments, when the longer, second end effector 600 is engaged with handle assembly 200 via the adapter assembly 400, the distance travelled by the firing rod 210 in response to actuation of the actuation switch 204 corresponds to the distance travelled by the drive assembly of the second end effector 600. That is, the distal end of the drive assembly of the second end effector 600 is configured to reach a distal-most end of its associated jaw member when the firing rod 210 reaches its distal-most position. Here, the biasing element 436 of the adapter assembly 430 does not significantly compress.

Accordingly, the adapter assembly 400 allows end effectors of differing lengths to be reliably used with the handle assembly 200 that is configured to advance the firing rod 210 a fixed or non-adjustable distance.

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A surgical kit, comprising:
a handle assembly including:
a handle portion;
an elongated portion extending distally from the handle portion;
an actuation switch; and
a firing rod, at least a portion of the firing rod extending through the elongated portion;
wherein actuation of the actuation switch causes the firing rod to move distally relative to the handle portion;
an adapter assembly configured to selectively engage the handle assembly, the adapter assembly including:
a connector, a proximal portion of the connecter configured to engage a distal portion of the firing rod of the handle assembly;
an adapter drive rod, a proximal portion of the adapter drive rod is coupled to a distal portion of the connector; and
a biasing element disposed between a distal end of the connector and a portion of the adapter drive rod; and
a first end effector defining a first length and configured to selectively engage the adapter assembly, the first end effector including:
a drive assembly, a proximal portion of the drive assembly configured to engage a distal portion of the adapter drive rod; and
a jaw member.

2. The surgical kit according to claim 1, wherein the adapter assembly and the handle assembly are configured such that when the adapter assembly is engaged with the handle assembly and with the first end effector, actuation of the actuation switch causes a first distal movement of the firing rod, a corresponding first distal movement of the adapter drive rod, and a corresponding first distal movement of the drive assembly of the first end effector to a distal-most position relative to the jaw member.

3. The surgical kit according to claim 2, wherein the handle assembly is configured such that the actuation of the actuation switch causes a corresponding first distal movement of the connector of the adapter assembly.

4. The surgical kit according to claim 3, wherein the handle assembly is configured such that the actuation of the actuation switch causes a second distal movement of the firing rod, a corresponding second distal movement of the connector of the adapter assembly, and results in the drive assembly of the first end effector remaining in its longitudinal position.

5. The surgical kit according to claim 4, wherein the adapter assembly is configured such that the adapter drive rod remains in its longitudinal position in response to the second distal movement of the firing rod.

6. The surgical kit according to claim 5, wherein the adapter assembly and the handle assembly are configured such that the second distal movement of the firing rod causes the biasing element to compress the amount of the second distal movement of the firing rod.

7. The surgical kit according to claim 1, wherein the biasing element of the adapter assembly is disposed between the distal end of the connector and a proximal-facing shoulder of the adapter drive rod.

8. The surgical kit according to claim 1, further comprising a second end effector defining a second length and configured to selectively engage the adapter assembly, wherein the second length is different from the first length.

9. The surgical kit according to claim 1, wherein the adapter assembly is configured such that the connector of the adapter assembly is longitudinally translatable relative to the drive assembly of the first end effector.

10. The surgical kit according to claim 1, wherein the adapter assembly and the handle assembly are configured such that when the adapter assembly is engaged with the handle assembly, the connector is longitudinally fixed relative to the firing rod.

11. The surgical kit according to claim 1, wherein the biasing element of the adapter assembly is a compression spring.

12. A surgical instrument, comprising:
a handle assembly including an actuation switch and a firing rod;
an adapter assembly configured to selectively engage the firing rod, the adapter assembly including a connector, an adapter drive rod, and a biasing element, the connector configured to engage a distal portion of the firing rod of the handle assembly, the biasing element configured to bias the adapter drive rod distally relative to the connector; and
an end effector configured to selectively engage the adapter assembly, the end effector including a drive assembly and a jaw member, the drive assembly configured to engage a distal portion of the adapter drive rod;
wherein actuation of the actuation switch causes the firing rod and the connector to move distally.

13. The surgical instrument according to claim 12, wherein initial actuation of the actuation switch causes the drive assembly of the end effector to move distally.

14. The surgical instrument according to claim 12, wherein the connector of the adapter assembly is longitudinally translatable relative to the drive assembly of the end effector.

15. The surgical instrument according to claim 12, wherein the biasing element is disposed between a distal end of the connector and a proximal-facing shoulder of the adapter drive rod.

16. The surgical instrument according to claim 12, wherein when the adapter assembly is engaged with the handle assembly, the connector is longitudinally fixed relative to the firing rod.

17. The surgical instrument according to claim 12, wherein the biasing element is a compression spring.

18. An adapter assembly for interconnecting a handle assembly of a surgical instrument and an end effector of a surgical instrument, the adapter assembly comprising:
a connector configured to engage a distal portion of a firing rod of the handle assembly;
an adapter drive rod disposed in mechanical cooperation with a distal portion of the connector; and
a biasing element disposed between a distal-most end of the connector and a portion of the adapter drive rod.

19. The adapter assembly according to claim 18, wherein the biasing element and the adapter drive rod are co-axial.

20. The adapter assembly according to claim 18, wherein the biasing element is a compression spring.
